(19) United States
(12) Patent Application Publication
(10) Pub. No.: US 2017/0296146 A1
(43) Pub. Date: Oct. 19, 2017

(54) ULTRASONIC DIAGNOSTIC DEVICE AND METHOD OF GENERATING DISCRIMINATION INFORMATION

(71) Applicant: Toshiba Medical Systems Corporation, Otawara-shi (JP)

(72) Inventor: Yuri HAYAKAWA, Nasushiobara (JP)

(73) Assignee: Toshiba Medical Systems Corporation, Otawara-shi (JP)

(21) Appl. No.: 15/487,950

(22) Filed: Apr. 14, 2017

(30) Foreign Application Priority Data
Apr. 19, 2016 (JP) 2016-083892

(51) Int. Cl. A61B 8/08; A61B 8/00; A61B 8/14; A61B 8/06

(52) U.S. Cl. CPC A61B 8/488; A61B 8/5207; A61B 8/461; A61B 8/06; A61B 8/4444; A61B 8/14; A61B 8/4483

(57) ABSTRACT

An ultrasonic diagnostic device according to an embodiment includes transmission/reception circuitry and processing circuitry. The transmission/reception circuitry intermittently executes first ultrasonic scanning on a first scanning region of a subject in accordance with a first scanning condition for generating a color Doppler image, and intermittently executes second ultrasonic scanning on a second scanning region of the subject in accordance with a second scanning condition for generating a form image. The processing circuitry generates a plurality of color Doppler images based on first echo data collected through the first ultrasonic scanning, generates a plurality of form images based on second echo data collected through the second ultrasonic scanning, and generates discrimination information for discriminating, from another color Doppler image, a color Doppler image related to a time phase in which a temporal change in luminance distribution of each of the form images is relatively large or small.

### CROSS-REFERENCE TO RELATED APPLICATIONS

This application is based upon and claims the benefit of priority from Japanese Patent Application No. 2016-083892, filed on Apr. 19, 2016; the entire contents of which are incorporated herein by reference.

### FIELD

Embodiments described herein relate generally to an ultrasonic diagnostic device and a method of generating discrimination information.

### BACKGROUND

There is known an ultrasonic diagnostic device having a function of generating and displaying blood flow information based on reflected waves of ultrasonic waves using the Doppler method based on the Doppler effect. In recent years, developed is a technique of generating a color Doppler image obtained by visualizing blood flow information in which a clutter component derived from a slowly moving tissue is greatly suppressed by visualizing a blood flow with high speed, high resolution, and a high frame rate. This color Doppler image is viewed by a user such as a doctor in diagnosing a subject.

### BRIEF DESCRIPTION OF THE DRAWINGS

FIG. 1 is a block diagram illustrating a configuration example of an ultrasonic diagnostic device according to a first embodiment;

FIG. 2 is a diagram illustrating a configuration example of the Doppler processing circuitry according to the first embodiment;

FIG. 3 is a diagram for explaining an example processing performed by an MTI filter;

FIG. 4 is a diagram for explaining an example of first ultrasonic scanning and second ultrasonic scanning according to the first embodiment;

FIG. 5 is a diagram for explaining another example of the first ultrasonic scanning and the second ultrasonic scanning according to the first embodiment;

FIG. 6 is a diagram for explaining an example of processing performed by processing circuitry according to the first embodiment;

FIG. 7A is a diagram illustrating an example of a display form according to the first embodiment;

FIG. 7B is a diagram illustrating an example of the display form according to the first embodiment;

FIG. 8 is a flowchart for explaining an example of ultrasonic scanning control processing performed by the ultrasonic diagnostic device according to the first embodiment;

FIG. 9 is a diagram for explaining an example of discrimination information according to a second embodiment; and

FIG. 10 is a diagram for explaining an example of discrimination information according to a third embodiment.

### DETAILED DESCRIPTION

An ultrasonic diagnostic device according to an embodiment includes transmission reception circuitry and processing circuitry. The transmission/reception circuitry intermittently performs first ultrasonic scanning on a first scanning region of a subject in accordance with a first scanning condition for generating a color Doppler image, and intermittently performs second ultrasonic scanning on a second scanning region of the subject in accordance with a second scanning condition for generating a form image. The processing circuitry generates a plurality of color Doppler images based on first echo data collected through the first ultrasonic scanning, generates a plurality of form images based on second echo data collected through the second ultrasonic scanning, and generates discrimination information for discriminating, from another color Doppler image, a color Doppler image related to a time phase in which a temporal change in luminance distribution of each of the form images is relatively large or a time phase in which the temporal change is relatively small.

The following describes the ultrasonic diagnostic device and a method of generating discrimination information according to the embodiment with reference to the drawings.

### First Embodiment

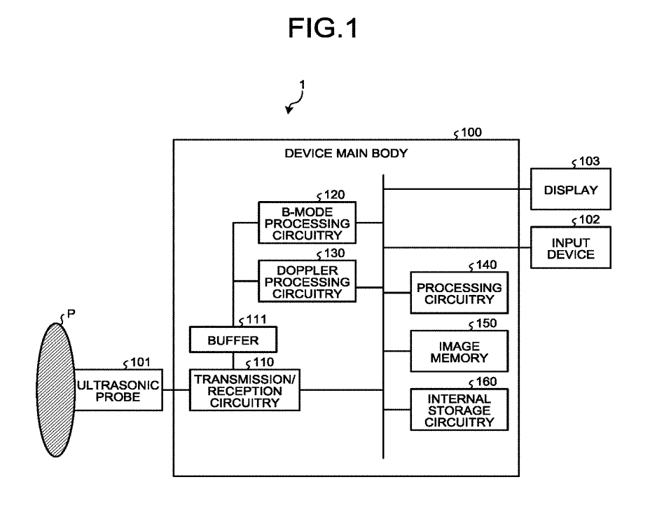

FIG. 1 is a block diagram illustrating a configuration example of an ultrasonic diagnostic device according to a first embodiment. As illustrated in FIG. 1, an ultrasonic diagnostic device 1 according to the first embodiment includes an ultrasonic probe 101, an input device 102, a display 103, and a device main body 100. The ultrasonic probe 101, the input device 102, and the display 103 are connected to the device main body 100 in a communicable manner. A subject. P is not included in the configuration of the ultrasonic diagnostic device 1.

The ultrasonic probe 101 transmits receives ultrasonic waves. For example, the ultrasonic probe 101 includes a plurality of piezoelectric transducer elements. The piezoelectric transducer elements generate ultrasonic waves based on a drive signal supplied from transmission reception circuitry 110 (described later) included in the device main body 100. The piezoelectric transducer elements included in the ultrasonic probe 101 receive reflected waves from the subject P, and convert the reflected waves into electric signals. The ultrasonic probe 101 includes a matching layer arranged in the piezoelectric transducer element, and a backing material that prevents the ultrasonic waves from propagating rearward from the piezoelectric transducer element. The ultrasonic probe 101 is detachably connected to the device main body 100.

When the ultrasonic waves are transmitted from the ultrasonic probe 101 to the subject P, the transmitted ultrasonic waves are successively reflected by a discontinuous surface of acoustic impedance in body tissues of the subject P, and received as reflected wave signals by the piezoelectric transducer elements included in the ultrasonic probe 101. Amplitude of the received reflected wave signal depends on a difference in acoustic impedance on the discontinuous surface that reflects the ultrasonic waves. The reflected wave signal obtained when a transmitted ultrasonic pulse is reflected by a surface of a moving blood flow, a cardiac wall, and the like is subjected to a frequency shift depending on a speed component with respect to an ultrasonic wave transmitting direction of a mobile object due to the Doppler effect.

The ultrasonic probe 101 according to the first embodiment can be applied to both of a 1D array probe that two-dimensionally scans the subject P and a mechanical 4D probe or a 2D array probe that three-dimensionally scans the subject P.

The input device 102 corresponds to a device such as a mouse, a keyboard, a button, a panel switch, a touch command screen, a foot switch, a trackball, a dial, a joystick, and a freeze button. The input device 102 receives various setting requests from a user of the ultrasonic diagnostic device 1, and transfers the received various setting requests to the device main body 100.

The display 103 displays a graphical user interface (GUI) for the user of the ultrasonic diagnostic device 1 to input various setting requests using the input device 102, and displays a B-mode image, a color Doppler, and the like generated by the device main body 100. For example, the display 103 is implemented by a liquid crystal monitor, a cathode ray tube (CRT) monitor, and a touch panel.

The device main body 100 is a device that generates ultrasonic image data based on the reflected wave signal received by the ultrasonic probe 101. The ultrasonic image data generated by the device main body 100 illustrated in FIG. 1 may be two-dimensional ultrasonic image data generated based on a two-dimensional reflected wave signal, or may be a three-dimensional ultrasonic image data generated based on a three-dimensional reflected wave signal.

As exemplified in FIG. 1, the device main body 100 includes the transmission/reception circuitry 110, B-mode processing circuitry 120, Doppler processing circuitry 130, processing circuitry 140, an image memory 150, and internal storage circuitry 160. The transmission/reception circuitry 110, the B-mode processing circuitry 120, the Doppler processing circuitry 130, the processing circuitry 140, the image memory 150, and the internal storage circuitry 160 are connected to each other in a communicable manner.

The transmission/reception circuitry 110 controls transmission and reception of ultrasonic waves performed by the ultrasonic probe 101 based on an instruction from the processing circuitry 140. For example, based on the instruction from the processing circuitry 140, the transmission/reception circuitry 110 according to the first embodiment controls transmission and reception of ultrasonic waves performed by the ultrasonic probe 101 so that first ultrasonic scanning is intermittently performed on a first scanning region of the subject P in accordance with a first scanning condition for generating a color Doppler image. Additionally, based on the instruction from the processing circuitry 140, the transmission/reception circuitry 110 controls transmission and reception of ultrasonic waves performed by the ultrasonic probe 101 so that second ultrasonic scanning is intermittently performed on a second scanning region of the subject P in accordance with a second scanning condition for generating a B-mode image (form image). That is, the transmission/reception circuitry 110 intermittently performs the first ultrasonic scanning on the first scanning region of the subject P in accordance with the first scanning condition for generating a color Doppler image via the ultrasonic probe 101. The transmission/reception circuitry 110 intermittently performs the second ultrasonic scanning on the second scanning region of the subject P in accordance with the second scanning condition for generating a B-mode image via the ultrasonic probe 101.

The transmission/reception circuitry 110 includes a pulse generator, transmission delay circuitry, and a pulser and supplies a drive signal to the ultrasonic probe 101. The pulse generator repeatedly generates a rate pulse for forming transmission ultrasonic waves at a predetermined pulse repetition frequency (PRF). The transmission delay circuitry converges the ultrasonic waves generated from the ultrasonic probe 101 as a beam, and gives, to each rate pulse generated by the pulse generator, a delay time for each piezoelectric transducer element required for determining transmission directivity. The pulser applies the drive signal (drive pulse) to the ultrasonic probe 101 at a timing based on the rate pulse. That is, the transmission delay circuitry optionally adjusts the transmitting direction of the ultrasonic waves transmitted from a surface of the piezoelectric transducer element by changing the delay time given to each rate pulse.

The transmission/reception circuitry 110 has a function of instantly changing a transmission frequency, a transmission driving voltage, and the like to execute a predetermined scanning sequence based on the instruction from the processing circuitry 140. Specifically, the transmission driving voltage is changed by a linear amplifier type transmission circuitry that can instantly switch a value thereof or a mechanism that electrically switches a plurality of power supply units.

The transmission/reception circuitry 110 includes amplifier circuitry, an analog/digital (A/D) converter, reception delay circuitry, an adder, and quadrature detection circuitry, and performs various pieces of processing on the reflected wave signal received by the ultrasonic probe 101 to generate reflected wave data (echo data).

The amplifier circuitry amplifies the reflected wave signal for each channel, and performs gain correction processing. The A/D converter A/D-converts the gain-corrected reflected wave signal. The reception delay circuitry gives, to digital data, a reception delay time required for determining reception directivity. The adder performs addition processing of the reflected wave signal to which the reception delay time is given by the reception delay circuitry. Through the addition processing performed by the adder, a reflection component from a direction corresponding to the reception directivity of the reflected wave signal is emphasized.

The quadrature detection circuitry converts output signals from the adder into a baseband in-phase signal (I signal, I: In-phase) and a baseband quadrature-phase signal (Q signal, Q: Quadrature-phase). The quadrature detection circuitry stores the I signal and the Q signal (hereinafter, referred to as an IQ signal) in a buffer 111 as the reflected wave data. The quadrature detection circuitry may convert the output signal from the adder into a radio frequency (RF) signal to be stored in the buffer 111. Each of the IQ signal and the RF signal is a signal (reception signal) including phase information.

The buffer 111 is a buffer that temporarily stores the reflected wave data (IQ signal) generated by the transmission/reception circuitry 110. Specifically, the buffer 111 stores IQ signals corresponding to several frames, or IQ signals corresponding to several volumes. For example, the buffer 111 is a first-in/first-out (FIFO) memory that stores IQ signals corresponding to predetermined frames. For example, when another IQ signal corresponding to one frame is generated by the transmission/reception circuitry 110, the buffer ill discards an IQ signal corresponding to one frame the generation time of which is the oldest, and stores the IQ signal corresponding to one frame that is newly generated. The buffer 111 is connected to the transmission/reception circuitry 110, the B-mode processing circuitry 120, and the Doppler processing circuitry 130 in a communicable manner.

The transmission/reception circuitry 110 can generate reflected wave data of a plurality of reception focuses from the reflected wave signal of each piezoelectric transducer element obtained through one time of transmission of an ultrasonic beam. That is, the transmission/reception circuitry 110 is circuitry that can perform parallel simultaneous reception processing. The first embodiment can be applied even when the transmission/reception circuitry 110 cannot perform parallel simultaneous reception processing.

Each of the B-mode processing circuitry 120 and the Doppler processing circuitry 130 is a processor that performs various pieces of signal processing on the reflected wave data generated from the reflected wave signal by the transmission/reception circuitry 110. The B-mode processing circuitry 120 performs logarithmic amplification, envelope detection processing, logarithmic compression, and the like on the reflected wave data read out from the buffer 111 to generate B-mode data in which multipoint signal intensity is expressed as luminance.

The B-mode processing circuitry 120 can change a frequency band to be visualized by changing a detection frequency through filter processing. By using a filter processing function of the B-mode processing circuitry 120, harmonic imaging such as contrast harmonic imaging (CHI) and tissue harmonic imaging (THI) can be executed.

By using the filter processing function of the B-mode processing circuitry 120, the ultrasonic diagnostic device 1 according to the first embodiment can execute tissue harmonic imaging (THI).

In executing harmonic imaging such as CHI and THI, the B-mode processing circuitry 120 can extract a harmonic component using a method different from the method using the filter processing described above. In harmonic imaging, used is an imaging method such as an amplitude modulation (AM) method, a phase modulation (PM) method, and an AMPM method combining the AM method and the PM method. In the AM method, the PM method, and the AMPM method, ultrasonic wave transmission is performed with respect to the same scanning line multiple times with different amplitude and different phases. Accordingly, the transmission/reception circuitry 110 generates and outputs a plurality of pieces of reflected wave data on each scanning line. The B-mode processing circuitry 120 performs addition and subtraction processing on the pieces of reflected wave data of each scanning line in accordance with the modulation method to extract the harmonic component. The B-mode processing circuitry 120 performs envelope detection processing and the like on the reflected wave data of the harmonic component to generate the B-mode data.

The Doppler processing circuitry 130 performs frequency analysis on the reflected wave data read out from the buffer 111 to generate Doppler data obtained by extracting motion information based on the Doppler effect of a mobile object within a scanning range. Specifically, the Doppler processing circuitry 130 generates the Doppler data obtained by estimating average speed, an average variance, and the like as the motion information of the mobile object at each of a plurality of sample points. In this case, the mobile object is, for example, a blond flow, a tissue such as a cardiac wall, and a contrast medium. Examples of the blood flow include an intracardiac blood flow and a blood flow within the cardiac wall. The Doppler processing circuitry 130 according to the present embodiment generates the Doppler data obtained by estimating average speed of the blood flow, an average variance of the blood flow, and the like as the motion information of the blood flow (blood flow information) at each of a plurality of sample points.

By using the function of the Doppler processing circuitry 130 described above, the ultrasonic diagnostic device 1 according to the present embodiment can execute a color Doppler method, which is also called a color flow mapping (CFM) method. In the CFM method, transmission and reception of ultrasonic waves are performed multiple times on a plurality of scanning lines. In the CFM method, a moving target indicator (MTI) filter is applied to a data column at the same position to suppress a signal (clutter signal) derived from a static tissue or a slowly moving tissue, and a signal derived from the blood flow is extracted. That is, in the CFM method, a blood flow component derived from the blood flow is extracted from the data column at the same position by suppressing the clutter component derived from the slowly moving tissue with the MTI filter. In the CFM method, the blood flow information such as speed of the blood flow and a variance of the blood flow is estimated based on a blood flow signal. The processing circuitry 140 (described later) generates a color Doppler image as an ultrasonic image in which distribution of estimation results is two-dimensionally color-displayed, for example. The Doppler processing circuitry 130 performs filter processing in a frame direction on a plurality of data columns of the reflected wave data at the same position of a plurality of frames to collect the blood flow information.

As the MTI filter, typically used is a filter the coefficient of which is fixed such as a Butterworth type infinite impulse response (IIR) filter and a polynomial regression filter. On the other hand, the Doppler processing circuitry 130 according to the present embodiment includes, as the MTI filter, an adaptive MTI filter the coefficient of which is changed in accordance with an input signal. Specifically, the Doppler processing circuitry 130 according to the present embodiment includes, as the adaptive MTI filter, a filter called an “eigenvector regression filter”. The “eigenvector regression filter” as the adaptive 1011 filter using an eigenvector is also called an “eigenvector MTI filter”.

The eigenvector MTI filter calculates an eigenvector from a correlation matrix, and calculates a coefficient to be used for clutter component suppression processing from the calculated eigenvector. This method is an application of a method used for principal component analysis, Karhunen-Loeve transform, and an eigenspace method.

The Doppler processing circuitry 130 according to the first embodiment including the eigenvector MTI filter calculates a correlation matrix of the scanning range from a continuous data column of the reflected wave data at the same position (same sample point). For example, the Doppler processing circuitry 130 calculates an eigenvalue of the correlation matrix and an eigenvector corresponding to the eigenvalue. The Doppler processing circuitry 130 calculates a matrix obtained by lowering a rank of the matrix in which each eigenvector is arranged based on magnitude of each eigenvalue, for example, as a filter matrix for suppressing the clutter component. The Doppler processing circuitry 130 determines, for example, the number of principal components to be reduced, that is, a value of the number of ranks to be cut based on a preset value or a value designated by the user. However, when the scanning range includes a tissue such as a heart and a blood vessel the moving speed of which varies with the lapse of time due to pulsation, it is preferable that the value of the number of ranks to be cut is adaptively determined based on magnitude of the eigenvalue. That is, the Doppler processing circuitry 130 changes the number of principal components to be reduced depending on magnitude of the eigenvalue of the correlation matrix. In the present embodiment, the Doppler processing circuitry 130 chances the number of ranks to be reduced depending on magnitude of the eigenvalue.

The Doppler processing circuitry 130 generates a data column obtained by suppressing the clutter signal and extracting the blood flow signal from the continuous data column of the reflected wave data at the same position using the filter matrix. The Doppler processing circuitry 130 performs an arithmetic operation such as an auto correlation arithmetic operation using the generated data to estimate the blood flow information, and outputs the estimated blood flow information as the Doppler data.

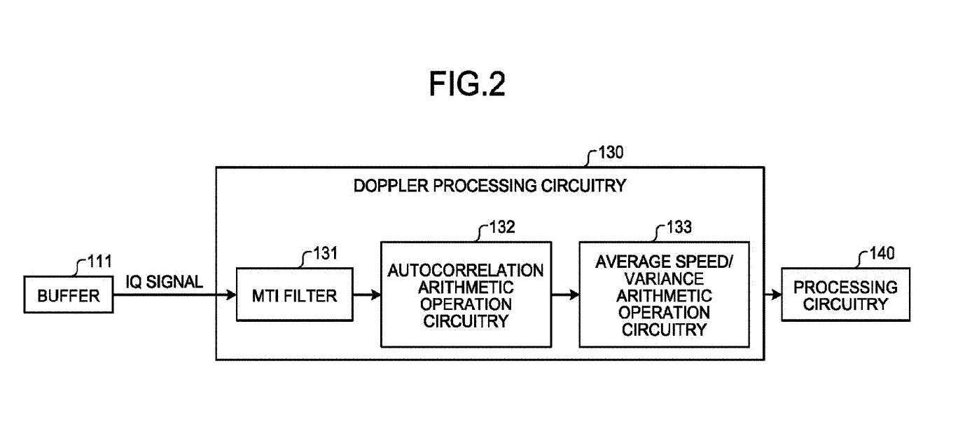

FIG. 2 is a diagram illustrating a configuration example of the Doppler processing circuitry according to the first embodiment. As illustrated in the example of FIG. 2, the Doppler processing circuitry 130 includes an MTI filter 131, autocorrelation arithmetic operation circuitry 132, and average speed/variance arithmetic operation circuitry 133.

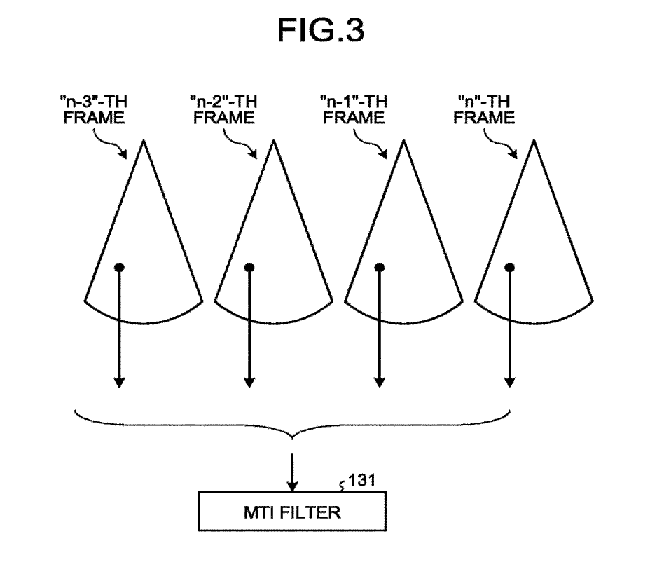

FIG. 3 is a diagram for explaining an example of filter processing performed by the Mil filter. As illustrated in the example of FIG. 3, to obtain filter output data (a blood flow signal) for the “n”-th frame, the MTI filter 131 uses reflected wave data (a reception signal) of the “n”-th frame, reflected wave data (a reception signal) of past three frames (the “n−3”-th frame to the “n−1”-th frame), and filter output data (a blood flow signal) of the past three frames at the same position. As described later, these pieces of reflected wave data are pieces of reflected wave data generated by performing transmission and reception of ultrasonic waves once for each of a plurality of scanning lines that form a scanning range (first scanning region) of one frame. Through the filter processing with the MTI filter 131, the blood flow signal from which the clutter signal is removed is extracted with high accuracy. For example, data is infinitely and continuously input to the MTI filter 131, so that a transient response is not generated in the filter processing. For example, the “n”-th frame corresponds to the n-th reflected wave data collected in the first ultrasonic scanning that is started when the ultrasonic diagnostic device 1 receives a scanning start request from the user.

Returning to FIG. 2, the autocorrelation arithmetic operation circuitry 132 takes a complex conjugate of the IQ signal of the blood flow signal of the latest frame and the IQ signal of the blood flow signal of a previous frame to calculate an autocorrelation value.

The average speed/variance arithmetic operation circuitry 133 calculates average speed and variance from the autocorrelation value calculated by the autocorrelation arithmetic operation circuitry 132. The average speed/variance arithmetic operation circuitry 133 then outputs the average speed and the variance as the Doppler data to the processing circuitry 140.

The Doppler processing circuitry 130 may further include power arithmetic operation circuitry, power addition circuitry, and logarithmic compression circuitry. The power arithmetic operation circuitry calculates power by adding a square of an absolute value of a real part of the IQ signal of the blood flow signal to a square of an absolute value of an imaginary part thereof. A value of the power shows scattering intensity caused by a reflector (for example, blood cell) smaller than a wavelength of the transmission ultrasonic wave. The power addition circuitry adds power at respective points together across optional frames. The logarithmic compression circuitry performs logarithmic compression on an output from the power addition circuitry, and outputs, as the Doppler data, the output from the power addition circuitry on which logarithmic compression is performed.

Returning to FIG. 1, the processing circuitry 140 has a function of generating various images and a function of controlling the entire processing of the ultrasonic diagnostic device 1. First, the following describes the function of generating various images. The processing circuitry 140 generates a plurality of color Doppler images based on the reflected wave data collected through the first ultrasonic scanning. The processing circuitry 140 generates a plurality of B-mode images based on the reflected wave data collected through the second ultrasonic scanning. The reflected wave data collected through the first ultrasonic scanning is an example of first reflected wave data (first echo data). The reflected wave data collected through the second ultrasonic scanning is an example of second reflected wave data (second echo data).

For example, the processing circuitry 140 generates an ultrasonic image from data generated by the B-mode processing circuitry 120 and the Doppler processing circuitry 130. As a specific example, the processing circuitry 140 generates a two-dimensional B-mode image in which intensity of the reflected wave is expressed as luminance from two-dimensional B-mode data generated by the B-mode processing circuitry 120. The processing circuitry 140 generates a two-dimensional Doppler image in which the blood flow information is visualised from two-dimensional Doppler data generated by the Doppler processing circuitry 130. The two-dimensional Doppler image is a speed image, a variance image, or a combination thereof. The processing circuitry 140 generates, as the Doppler image, color Doppler image data in which the blood flow information is color-displayed, or generates Doppler image data in which a piece of blood flow information is displayed with a gray scale.

Typically, the processing circuitry 140 converts (scan-converts) a scanning line signal string of ultrasonic scanning into a scanning line signal string of a video format represented by a television and the like, and generates an ultrasonic image data for display. Specifically, the processing circuitry 140 performs coordinate transformation in accordance with a mode of scanning the ultrasonic waves by the ultrasonic probe 101 to generate the ultrasonic image data for display. In addition to the scan-conversion, the processing circuitry 140 performs, as various pieces of image processing, image processing (smoothing processing) for regenerating an average value image of luminance, image processing (edge emphasis processing) using a differential filter in an image, and the like using a plurality of image frames after the scan-conversion, for example. The processing circuitry 140 synthesizes the ultrasonic image data with character information of various parameters, a scale, a body mark, and the like.

That is, the B-mode data and the Doppler data are ultrasonic image data before scan-conversion processing, and the data generated by the processing circuitry 140 is the ultrasonic image data for display after scan-conversion processing. Each of the B-mode data and the Doppler data is also called raw data. The processing circuitry 140 generates a two-dimensional ultrasonic image for display from two-dimensional ultrasonic image data before scan-conversion processing.

Additionally, the processing circuitry 140 performs coordinate transformation on three-dimensional B-mode data generated by the B-mode processing circuitry 120 to generate a three-dimensional B-mode image. The processing circuitry 140 performs coordinate transformation on three-dimensional Doppler data generated by the Doppler processing circuitry 130 to generate a three-dimensional Doppler image.

To generate various two-dimensional images for displaying volume data on the display 103, the processing circuitry 140 performs rendering processing on the volume data. Examples of the rendering processing performed by the processing circuitry 140 include processing of generating an MPR image from the volume data by performing multi planer reconstruction (MPR). Examples of the rendering processing performed by the processing circuitry 140 also include volume rendering (VR) processing of generating a two-dimensional image in which three-dimensional information is reflected.

Next, the following describes the function of controlling the entire processing of the ultrasonic diagnostic device 1. For example, the processing circuitry 140 controls pieces of processing of the transmission/reception circuitry 110, the B-mode processing circuitry 120, and the Doppler processing circuitry 130 based on various setting requests input by the user via the input device 102 and various control programs and various pieces of data read from the internal storage circuitry 160. The processing circuitry 140 controls the display 103 to display the ultrasonic image for display stored in the image memory 150 or the internal storage circuitry 160.

For example, the processing circuitry 140 controls the ultrasonic probe 101 via the transmission/reception circuitry 110 to control ultrasonic scanning. Typically, in the CFM method, the B-mode image as a tissue image data is displayed together with the color Doppler image as a blood flow image data. To perform such display, the processing circuitry 140 causes the ultrasonic probe 101 to execute the first ultrasonic scanning for acquiring the blood flow information in the first scanning region. The first ultrasonic scanning is, for example, ultrasonic scanning for collecting the color Doppler image data in a Doppler mode. The processing circuitry 140 also causes the ultrasonic probe 101 to execute the second ultrasonic scanning for acquiring information of a tissue shape in the second scanning region together with the first ultrasonic scanning. The second ultrasonic scanning is, for example, ultrasonic scanning for collecting B-mode image data in a B-mode.

Programs corresponding to the various pieces of processing described above performed by the processing circuitry 140 are stored in the internal storage circuitry 160 in a computer-executable manner. The processing circuitry 140 is a processor that reads out each program from the internal storage circuitry 160, and executes the read program to perform the various pieces of processing described above.

In the embodiment described above, the various pieces of processing described above are performed by a single processing circuitry 140. Alternatively, the processing circuitry may be configured by combining a plurality of independent processors, and each processor may execute the program to perform corresponding processing.

The word “processor” used in the above description means, for example, a central processing unit (CPU), a graphics processing unit (GPU), or circuitry such as an application specific integrated circuit (ASIC) and a programmable logic device (for example, a simple programmable logic device (SPLD), a complex programmable logic device (CPLD), and a field programmable gate array (FPGA)). The processor reads out and executes the program stored in the internal storage circuitry 160 to implement various functions. Instead of storing the program in the internal storage circuitry 160, the program may be directly incorporated in circuitry of the processor. In this case, the processor reads out and executes the program incorporated in the circuitry to implement various functions. Each processor according to the present embodiment is not necessarily configured as a single circuitry. A plurality of independent circuitries may be combined to be one processor to implement the function thereof. A plurality of components in each drawing may be integrated into one processor to implement the function thereof.

The image memory 150 is a memory that stores image data for display generated by the processing circuitry 140. The image memory 150 can also store the B-mode data generated by the B-mode processing circuitry 120 and the Doppler data generated by the Doppler processing circuitry 130. The B-mode data or the Doppler data stored in the image memory 150 can be called by the user after diagnosis, for example, and becomes an ultrasonic image for display via the processing circuitry 140. The image memory 150 can also store the reflected wave data output by the transmission/reception circuitry 110. The image memory 150 is an example of a storage circuitry.

The internal storage circuitry 160 stores a control program for performing transmission and reception of ultrasonic waves, image processing, and display processing, diagnostic information (for example, a patient ID and findings of a doctor), and various pieces of data such as a diagnostic protocol and various body marks. The internal storage circuitry 160 is also used, for example, for keeping the ultrasonic image generated by the processing circuitry 140 as needed. The data stored in the internal storage circuitry 160 can be transferred to an external device via an interface (not illustrated). The internal storage circuitry 160 can also store data transferred from an external device via an interface (not illustrated).

The following describes a case in which, for example, with the ultrasonic diagnostic device, the user sets a region of interest with respect to a vigorously moving tissue such as a cardiac wall, and tries to check the color Doppler image (myocardial perfusion image) in which a blood flow in a capillary of the tissue such as a cardiac wall is visualized. One heartbeat includes a time phase in which movement of the tissue such as the cardiac wall is vigorous (variation is relatively large), and a time phase in which the movement thereof is gentle (variation is relatively small). In the data column of the reflected wave data collected in the time phase in which the movement of the tissue is vigorous, a spectrum of the clutter component may be overlapped with a spectrum of the blood flow component in some cases. Thus, when the MTI filter is applied to such a data column of the reflected wave data, the blood flow component may be hardly separated from the clutter component as a noise component. Accordingly, the myocardial perfusion image is not a high-definition image in some cases, the myocardial perfusion image being generated based on the reflected wave data collected in the time phase in which the movement of the tissue is vigorous. It is hard to say that such a low-definition myocardial perfusion image is useful for diagnosing the subject.

On the other hand, in the data column of the reflected wave data collected in the time phase in which the movement of the tissue is gentle, the spectrum of the clutter component is highly likely not to be overlapped with the spectrum of the blood flow component. When the MTI filter is applied to such a data column of the reflected wave data, the blood flow component can be separated from the clutter component in some cases. Accordingly, the myocardial perfusion image is highly likely to be a high-definition image, the myocardial perfusion image being generated based on the reflected wave data collected in the time phase in which the movement of the tissue is gentle. Such a high-definition myocardial perfusion image is useful for diagnosing the subject.

As described above, a plurality of color Doppler images generated in accordance with one heartbeat include the color Doppler image useful for diagnosis and the color Doppler image that is not useful for diagnosis.

Thus, as described below, the ultrasonic diagnostic device 1 according to the first embodiment presents, to the user, information useful for diagnosis indicating that which color Doppler image is useful for diagnosis among a plurality of color Doppler images. Accordingly, the user can easily grasp that which color Doppler image is useful for diagnosis among a plurality of color Doppler images. Thus, convenience for the user to perform diagnosis can be enhanced.

The ultrasonic diagnostic device 1 according to the first embodiment visualizes the blood flow with high speed, high resolution, and a high frame rate to perform, as the first ultrasonic scanning, ultrasonic scanning for the Doppler mode for obtaining blood flow information in which the clutter component is significantly suppressed as compared with the typical Doppler method. For example, the first ultrasonic scanning is executed by repeating a scanning mode in which the reflected wave data at the same position can be collected across a plurality of frames through transmission and reception of ultrasonic waves in a scanning range including a plurality of scanning lines. More specifically, the first ultrasonic scanning according to the first embodiment is executed by repeating the scanning mode in which the ultrasonic waves are transmitted and received once for each scanning line in the scanning range including a plurality of scanning lines. This scanning mode is the same as that of ultrasonic scanning executed in the typical B-mode, and is the same as the scanning mode performed in the CFM method for improving the frame rate.

The ultrasonic diagnostic device 1 executes ultrasonic scanning in the second scanning region as the second ultrasonic scanning during the first ultrasonic scanning. Accordingly, in the first embodiment, a scanning condition can be independently set for the first ultrasonic scanning and the second ultrasonic scanning.

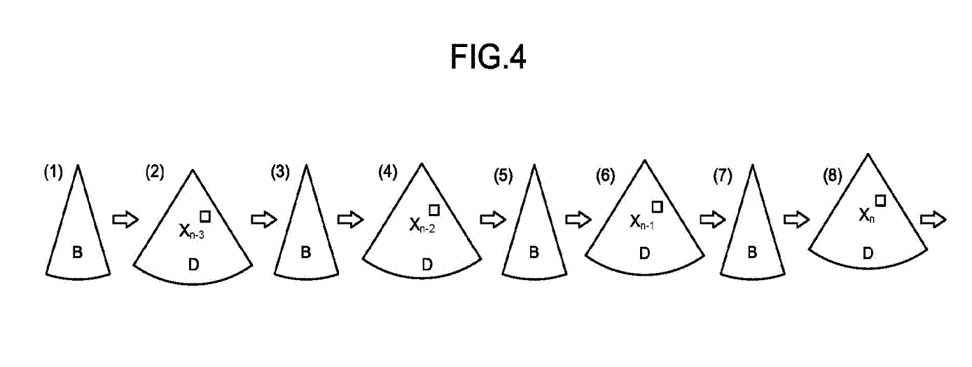

The following describes an example of the first ultrasonic scanning and the second ultrasonic scanning with reference to FIG. 4. FIG. 4 is a diagram for explaining an example of the first ultrasonic scanning and the second ultrasonic scanning according to the first embodiment. In FIG. 4, “B” represents the second scanning region in which ultrasonic scanning is performed using a transmission/reception condition for the B-mode. In FIG. 4, “D” represents the first scanning region in which ultrasonic scanning is performed using a transmission/reception condition for the color Doppler mode. For example, in the first ultrasonic scanning illustrated in FIG. 4, transmission and reception of ultrasonic waves are performed once for each scanning line instead of transmitting the ultrasonic waves multiple times in the same direction and receiving the reflected waves multiple times like the typical color Doppler method. That is, the processing circuitry 140 transmits, to the transmission/reception circuitry 110, an instruction to cause the ultrasonic probe 101 to execute ultrasonic scanning for collecting the Doppler image data of the blood flow as the first ultrasonic scanning. The processing circuitry 140 transmits, to the Doppler processing circuitry 130, an instruction to perform filter processing in the frame direction on the reflected wave data acquired from each of a plurality of scanning lines included in the first scanning region. The processing circuitry 140 according to the first embodiment performs transmission and reception of ultrasonic waves once for each of a plurality of scanning lines included in the first scanning region to acquire a reception signal for each of the scanning lines included in the first scanning region, and causes ultrasonic scanning for acquiring the data column in the frame direction in which filter processing is performed to be executed as the first ultrasonic scanning. That is, the processing circuitry 140 according to the first embodiment performs transmission and reception of ultrasonic waves once for each of a plurality of scanning lines included in the first scanning region as the first ultrasonic scanning, and causes the ultrasonic scanning to be executed for acquiring information related to motion of the mobile object using the reflected waves corresponding to a plurality of frames.

As illustrated in the example of FIG. 4, first, the processing circuitry 140 transmits, to the transmission/reception circuitry 110, an instruction to cause the ultrasonic probe 101 to execute ultrasonic scanning in the second scanning region as the second ultrasonic scanning. Accordingly, as illustrated in (1) of FIG. 4, the second ultrasonic scanning is executed. The B-mode processing circuitry 120 generates the B-mode data corresponding to one frame based on the reflected wave data corresponding to one frame obtained through the second ultrasonic scanning. When the B-mode data corresponding to one frame is generated, the processing circuitry 140 generates the B-mode image based on the generated B-mode data corresponding to one frame. In this way, the B-mode image corresponding to one frame is generated through one time of second ultrasonic scanning.

Next, the processing circuitry 140 transmits, to the transmission/reception circuitry 110, an instruction to cause the ultrasonic probe 101 to execute the first ultrasonic scanning in the first scanning region. Accordingly, as illustrated in (2) of FIG. 4, the first ultrasonic scanning is executed. The Doppler processing circuitry 130 generates the Doppler data corresponding to one frame based on the reflected wave data corresponding to one frame obtained through the first ultrasonic scanning. When the Doppler data corresponding to one frame is generated, the processing circuitry 140 generates, as the Doppler image, the color Doppler image in which the blood flow information is color-displayed or generates the Doppler image in which one piece of blood flow information is displayed with a gray scale based on the generated Doppler data corresponding to one frame. In this way, the Doppler image corresponding to one frame is generated through one time of first ultrasonic scanning.

The processing circuitry 140 transmits, to the transmission/reception circuitry 110, an instruction to cause the ultrasonic probe 101 to execute ultrasonic scanning in the second scanning region as the second ultrasonic scanning. Accordingly, as illustrated in (3) of BIG. 4, the second ultrasonic scanning is executed. As a result, another B-mode image corresponding to one frame is generated. The processing circuitry 140 then transmits, to the transmission/reception circuitry 110, an instruction to cause the ultrasonic probe 101 to execute the first ultrasonic scanning in the first scanning region. Accordingly, as illustrated in (4) of FIG. 4, the first ultrasonic scanning is executed. As a result, another Doppler image corresponding to one frame is generated.

The processing circuitry 140 then transmits, to the transmission/reception circuitry 110, an instruction to cause the ultrasonic probe 101 to execute ultrasonic scanning in the second scanning region as the second ultrasonic scanning. Accordingly, as illustrated in (5) of FIG. 4, the second ultrasonic scanning is executed. As a result, another B-mode image corresponding to one frame is generated. The processing circuitry 140 then transmits, to the transmission/reception circuitry 110, an instruction to cause the ultrasonic probe 101 to execute the first ultrasonic scanning in the first scanning region. Accordingly, as illustrated in (6) of FIG. 4, the first ultrasonic scanning is executed. As a result, another Doppler image corresponding to one frame is generated.

The processing circuitry 140 then transmits, to the transmission/reception circuitry 110, an instruction to cause the ultrasonic probe 101 to execute ultrasonic scanning in the second scanning region as the second ultrasonic scanning. Accordingly, as illustrated in (7) of FIG. 4, the second ultrasonic scanning is executed. As a result, another B-mode image corresponding to one frame is generated. The processing circuitry 140 then transmits, to the transmission/reception circuitry 110, an instruction to cause the ultrasonic probe 101 to execute the first ultrasonic scanning in the first scanning region. Accordingly, as illustrated in (8) of FIG. 4, the first ultrasonic scanning is executed. As a result, another Doppler image corresponding to one frame is generated.

As illustrated in the example of FIG. 4, a point X on a certain scanning line in the first scanning region is scanned once in each first ultrasonic scanning of (2), (4), (6), and (8). The Doppler processing circuitry 130 performs the filter processing described above on the data column (Xn-3, Xn-2, Xn-1, Xn) at the same position between “D” frames to output the motion information of the blood flow at the point X.

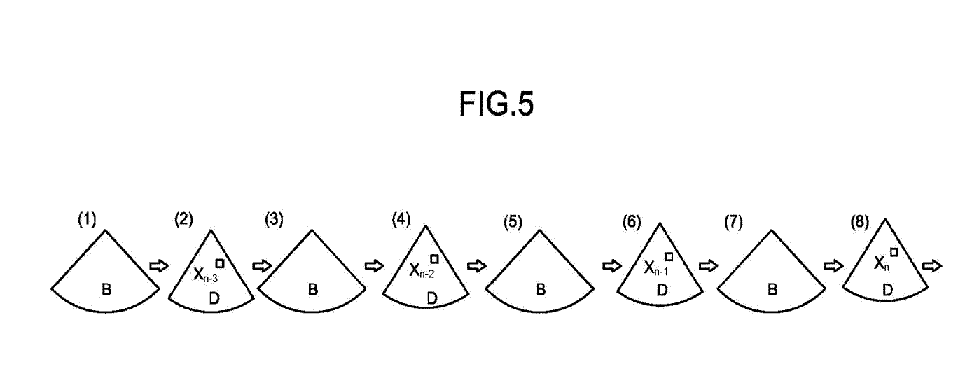

In the example of FIG. 4, described is a case in which the second scanning region is smaller than the first scanning region. Alternatively, as illustrated in the example of FIG. 5, the second scanning region may be larger than the first scanning region. FIG. 5 is a diagram for explaining another example of the first ultrasonic scanning and the second ultrasonic scanning according to the first embodiment. In the example of FIG. 5, described is a configuration in which the second scanning region is larger than the first scanning region. Other configurations are the same as those in the example of FIG. 4, so that redundant description will not be repeated. The size of the first scanning region may be the same as that of the second scanning region.

As described above, in the first embodiment, the scanning condition can be independently set for the first ultrasonic scanning and the second ultrasonic scanning. In this way, in the first embodiment, an optimum scanning condition for the B-mode can be set, and an optimum scanning condition for the color Doppler mode can be set. For example, in the first embodiment, an optimum scanning condition for THI such as the PM method can be set as a scanning condition for the second ultrasonic scanning. Thus, in the first embodiment, image quality of the color Doppler image (for example, the myocardial perfusion image described above) and the B-mode image can be improved, the color Doppler image and the B-mode image being displayed at the same time. The scanning condition for the second ultrasonic scanning may be different from a scanning condition for the first ultrasonic scanning in at least one of a frequency band of ultrasonic waves to be transmitted and a frequency band of ultrasonic waves to be received. For example, the frequency band of the ultrasonic waves to be transmitted included in an operation condition for the second ultrasonic scanning may be caused to be wider than the frequency band of ultrasonic waves to be transmitted included in an operation condition for the first ultrasonic scanning, and the frequency band of the ultrasonic waves to be received included in the operation condition for the second ultrasonic scanning may be caused to be wider than the frequency band of ultrasonic waves to be received included in the operation condition for the first ultrasonic scanning.

The processing circuitry 140 performs processing described below every time the B-mode image is generated. That is, the processing circuitry 140 calculates a temporal change in luminance distribution of adjacent two B-mode images in a time axis direction.

For example, the processing circuitry 140 calculates a difference between luminance of each pixel in a predetermined region in one of the adjacent two B-mode images and luminance of each pixel in a corresponding region in the other one of the adjacent two B-mode images. The predetermined region is a part or the entirety of the scanning region. In addition, the predetermined region is a region designated by the user's operation or a region automatically designated by image analysis on the B-mode image, for example. Hereinafter, the “difference between luminance of each pixel in a predetermined region in one B-mode image and luminance of each pixel in a corresponding region in the other B-mode image” may also be referred to as the “difference between the luminance of one B-mode image and the luminance of the other B-mode image”. The processing circuitry 140 calculates the sum of differences in luminance calculated for each pixel as the temporal change in luminance distribution of the adjacent two B-mode images. The processing circuitry 140 then determines whether the calculated sum of the differences in luminance is smaller than a predetermined threshold.

In this way, every time the B-mode image is generated, the processing circuitry 140 determines whether the sum of the differences in luminance between the most lately generated B-mode image and the B-mode image generated immediately prior to the former B-mode image is smaller than the predetermined threshold.

If it is determined that the sum of the differences in luminance is smaller than the predetermined threshold a predetermined number of times or more in a row, the processing circuitry 140 specifies a time phase corresponding to the B-mode image the sum of the differences in luminance of which is successively determined to be smaller than the predetermined threshold as a time phase in which the temporal change in luminance distribution is relatively small.

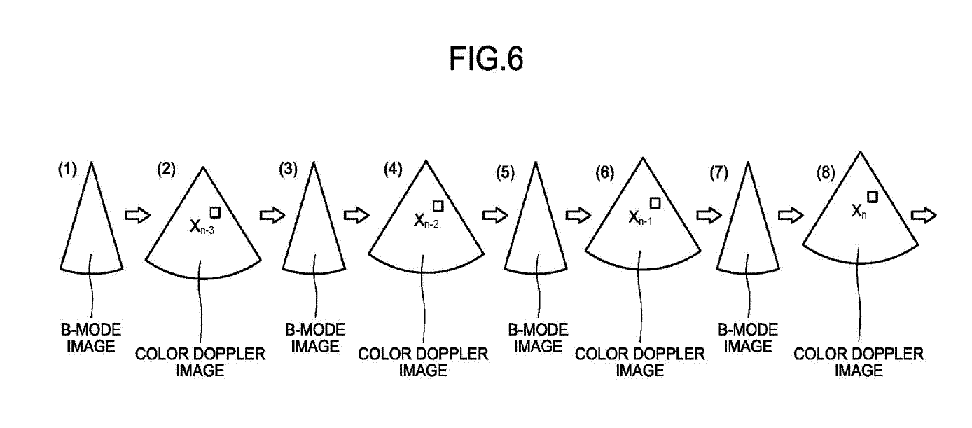

FIG. 6 is a diagram for explaining an example of processing performed by the processing circuitry according to the first embodiment. In FIG. 6, (1) illustrates the B-mode image generated based on the reflected wave data obtained through the second ultrasonic scanning illustrated in (1) of FIG. 4. In FIG. 6, (3) illustrates the B-mode image generated based on the reflected wave data obtained through the second ultrasonic scanning illustrated in (3) of FIG. 4. In FIG. 6, (5) illustrates the B-mode image generated based on the reflected wave data obtained through the second ultrasonic scanning illustrated in (5) of FIG. 4. In FIG. 6, (7) illustrates the B-mode image generated based on the reflected wave data obtained through the second ultrasonic scanning illustrated in (7) of FIG. 4.

In FIG. 6, (2) illustrates the color Doppler image generated based on the reflected wave data obtained through the first ultrasonic scanning illustrated in (2) of FIG. 4. In FIG. 6, (4) illustrates the color Doppler image generated based on the reflected wave data obtained through the first ultrasonic scanning illustrated in (4) of FIG. 4. In FIG. 6, (6) illustrates the color Doppler image generated based on the reflected wave data obtained through the first ultrasonic scanning illustrated in (6) of FIG. 4. In FIG. 6, (8) illustrates the color Doppler image generated based on the reflected wave data obtained through the first ultrasonic scanning illustrated in (8) of FIG. 4.

There is only one B-mode image at a stage when the B-mode image illustrated in (1) of FIG. 6 is generated, so that the processing circuitry 140 does not perform processing of calculating the temporal change in luminance distribution of the adjacent two B-mode images described above. When the B-mode image illustrated in (3) of FIG. 6 is generated, the processing circuitry 140 calculates the difference between the luminance of each pixel in a predetermined region in the B-mode image illustrated in (1) of FIG. 6 and the luminance of each pixel in a corresponding region in the B-mode image illustrated in (3) of FIG. 6. The processing circuitry 140 then calculates the sum of the differences in luminance calculated for each pixel as the temporal change in luminance distribution of the B-mode image illustrated in (1) of FIG. 6 and the B-mode image illustrated in (3) of FIG. 6. The processing circuitry 140 determines whether the calculated sum of the differences in luminance is smaller than the predetermined threshold.

When the B-mode image illustrated in (5) of FIG. 6 is generated, the processing circuitry 140 calculates the difference between the luminance of each pixel in a predetermined region in the B-mode image illustrated in (3) of FIG. 6 and the luminance of each pixel in a corresponding region in the B-mode image illustrated in (5) of FIG. 6. The processing circuitry 140 then calculates the sum of the differences in luminance calculated for each pixel as the temporal change in luminance distribution of the B-mode image illustrated in (3) of FIG. 6 and the B-mode image illustrated in (5) of FIG. 6. The processing circuitry 140 determines whether the calculated sum of the differences in luminance is smaller than the predetermined threshold.

When the B-mode image illustrated in (7) of FIG. 6 is generated, the processing circuitry 140 calculates the difference between the luminance of each pixel in a predetermined region in the B-mode image illustrated in (5) of FIG. 6 and the luminance of each pixel in a corresponding region in the B-mode image illustrated in (7) of FIG. 6. The processing circuitry 140 then calculates the sum of the differences in luminance calculated for each pixel as the temporal change in luminance distribution of the B-mode image illustrated in (5) of FIG. 6 and the B-mode image illustrated in (7) of FIG. 6. The processing circuitry 140 determines whether the calculated sum of the differences in luminance is smaller than the predetermined threshold.

The following describes a case in which the predetermined number of times described above is three, the predetermined number of times being used for specifying time phase in which the temporal change in luminance distribution is relatively small. In this case, if it is determined that the calculated sum of the differences in luminance is smaller than the predetermined threshold in the B-mode image illustrated in (1) of FIG. 6 and the B-mode image illustrated in (3) of FIG. 6, in the B-mode image illustrated in (3) of FIG. 6 and the B-mode image illustrated in (5) of FIG. 6, and in the B-mode image illustrated in (5) of FIG. 6 and the B-mode image illustrated in (7) of FIG. 6, that is, if it is determined that the sum of the differences in luminance is smaller than the predetermined threshold three times in a row, the processing circuitry 140 performs the following processing. That is, the processing circuitry 140 specifies, as the time phase in which the temporal change in luminance distribution is relatively small, a time phase in a range from the B-mode image illustrated in (1) of FIG. 6 to the B-mode image illustrated in (7) of FIG. 6 in which the sum of the differences in luminance is determined to be smaller than the predetermined threshold three times in a row. If the time phase in a range from the B-mode image illustrated in (1) of FIG. 6 to the B-mode image illustrated in (7) of FIG. 6 is specified as the time phase in which the temporal change in luminance distribution is relatively small, a time phase corresponding to the color Doppler image illustrated in (2) of FIG. 6, the color Doppler image illustrated in (4) of FIG. 6, and the color Doppler image illustrated in (6) of FIG. 6 also becomes the time phase in which the temporal change in luminance distribution is relatively small.

The time phase in which the temporal change in luminance distribution is relatively small is considered to be a time phase in which movement of tissues such as a cardiac wall is gentle, for example. Thus, the processing circuitry 140 can specify the time phase in which movement of tissues such as a cardiac wall is gentle by specifying the time phase in which the temporal change in luminance distribution is relatively small.

In subsequent processing, the processing circuitry 140 performs the same processing every time the B-mode image is generated until it is determined that the sum of the differences in luminance is equal to or larger than the predetermined threshold. If it is determined that the sum of the differences in luminance is equal to or larger than the predetermined threshold, the processing circuitry 140 specifies, as the time phase in which the temporal change in luminance distribution is relatively small, a time phase in a range from the B-mode image illustrated in (1) of FIG. 6 to the B-mode image the sum of the differences in luminance of which is lastly determined to be smaller than the predetermined threshold.

The processing circuitry 140 then generates discrimination information for discriminating a color Doppler image related to the specified time phase from another color Doppler image among a plurality of color Doppler images. For example, the following describes a case in which the time phase corresponding to the color Doppler image illustrated in (2) of FIG. 6, the color Doppler image illustrated in (4) of FIG. 6, and the color Doppler image illustrated in of FIG. 6 is the time phase in which the temporal change in luminance distribution is relatively small. In this case, the processing circuitry 140 generates an image indicating a red frame for discriminating, from another color Doppler image, the color Doppler image illustrated in (2) of FIG. 6, the color Doppler image illustrated in (4) of FIG. 6, and the color Doppler image illustrated in (6) of FIG. 6 among a plurality of color Doppler images. The processing circuitry 140 then synthesizes the image indicating the red frame with each of the color Doppler image illustrated in of FIG. 6, the color Doppler image illustrated in (4) of FIG. 6, and the color Doppler image illustrated in (6) of FIG. 6 so that the red frame indicated by the generated image surrounds each of the color Doppler image illustrated in (2) of FIG. 6, the color Doppler image illustrated in (4) of FIG. 6, and the color Doppler image illustrated in (6) of FIG. 6. Thus, when the color Doppler image related to the specified time phase is displayed, the image indicating the red frame is also displayed together with the color Doppler image.

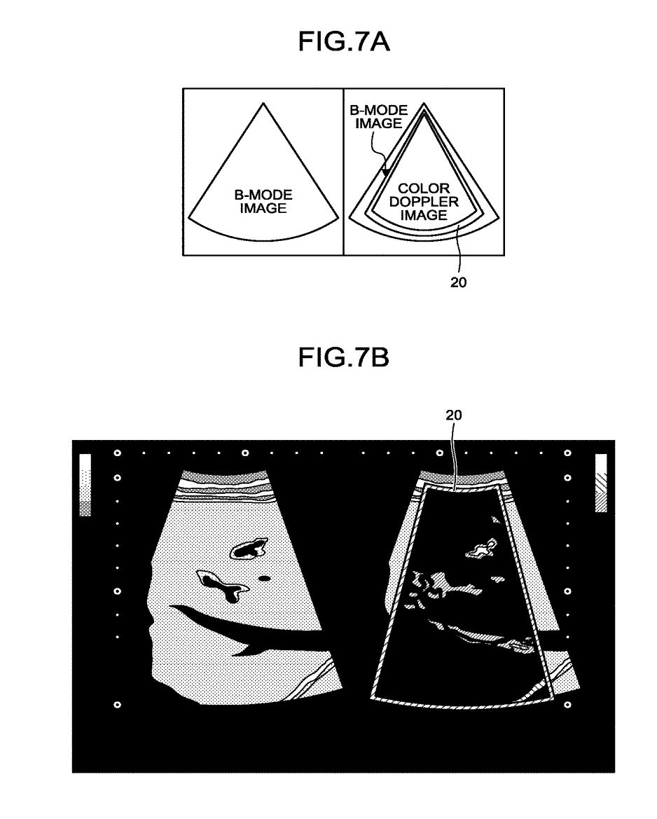

The processing circuitry performs display control as illustrated in FIGS. 7A and 7B, for example. FIGS. 7A and 7B are diagrams illustrating an example of a display form according to the first embodiment. FIG. 7A is a diagram for schematically illustrating a positional relation between the color Doppler image and the B-mode image on the display 103. For example, in a real-time display mode in which the B-mode image and the color Doppler image are displayed in real time, the processing circuitry 140 controls the display 103 to display the B-mode image on the left side and perform superimposed display such that the B-mode image is superimposed on the color Doppler image on the right side as illustrated in FIGS. 7A and 7B. That is, when the B-mode image is newly generated, the processing circuitry 140 updates the already displayed B-mode image with the newly generated B-mode image. When the color Doppler image is newly generated, the processing circuitry 140 updates the already displayed color Doppler image with the newly generated color Doppler image. In the example illustrated in FIGS. 7A and 7B, the first scanning region is set in the second scanning region.

For example, FIG. 7B illustrates a case in which the B-mode image illustrated in FIG. 7A is the B-mode image generated by THI, and the color Doppler image illustrated in FIG. 7A is the myocardial perfusion image described above. In this case, the myocardial perfusion image illustrated in FIG. 7B is a color Doppler image corresponding to the time phase in which the temporal change in luminance distribution is relatively small, so that an image 20 indicating the red frame is displayed together with the myocardial perfusion image. That is, the myocardial perfusion image is highlighted to be emphasized. The myocardial perfusion image illustrated in FIG. 7B is a color Doppler image corresponding to the time phase in which the temporal change in luminance distribution is relatively small, which is a high-quality image through which the user can check a flow of blood oozing out of a cardiac wall. In this way, the ultrasonic diagnostic device 1 according to the present embodiment presents, to the user, the image 20 as information indicating that the displayed myocardial perfusion image is a myocardial perfusion image useful for diagnosis. Accordingly, the user can easily grasp that which myocardial perfusion image is useful for diagnosis among a plurality of myocardial perfusion images. Thus, convenience for the user to perform diagnosis can be enhanced.

The B-mode image illustrated in FIGS. 7A and 7B may be a typical B-mode image. The color Doppler image illustrated in FIGS. 7A and 7B may be a color Doppler image other than the myocardial perfusion image.

The processing circuitry 140 may perform other display control in addition to the display control in real time. For example, when the user presses a freeze button in the real-time display mode, the processing circuitry 140 proceeds from the real-time display mode to a cine-reproduction mode. In the cine-reproduction mode, the processing circuitry 140 acquires the B-mode image and the color Doppler image stored in the image memory 150. When the trackball, the dial or the like is rotated by the user, the processing circuitry 140 reproduces the B-mode image and the color Doppler image on the display 103 in accordance with the rotational direction, rotation amount and the like of the trackball or the dial. Alternatively, the processing circuitry 140 dynamically reproduces the B-mode image and the color Doppler image on the display 103 without any rotation of the trackball, the dial or the like. The display form of the B-mode image and the color Doppler image is the same as the display form in the real-time display mode described above with reference to FIGS. 7A and 7B, for example. Accordingly, the user can easily grasp that which color Doppler image is useful for diagnosis among a plurality of color Doppler images also in the cine-reproduction mode. Thus, convenience for the user to perform diagnosis can be enhanced.

However, in the present embodiment, there is a difference between the real-time display mode and the cine-reproduction mode as follows. For example, in the real-time display mode, although the B-mode image and the color Doppler image can be displayed in real time, the user cannot easily grasp ail color Doppler images useful for diagnosis in some cases. This is because the processing circuitry 140 does not generate the information indicating that which color Doppler image is useful for diagnosis (in the above example, the image 20 indicating the red frame) until it is determined that the sum of the differences in luminance is smaller than the predetermined threshold the predetermined number of times in a row. For example, even when a time phase corresponding to a certain color Doppler image is the time phase in which the temporal change in luminance distribution is relatively small, the image 20 indicating the red frame is not synthesized with the color Doppler image at a stage when the processing circuitry 140 does not determine that the sum of the differences in luminance is smaller than the predetermined threshold the predetermined number of times or more in a row. Thus, in the real-time display mode, at the stage when the sum of the differences in luminance is not determined to be smaller than the predetermined threshold the predetermined number of times or more in a row, the color Doppler image useful for diagnosis may be displayed as it is without being synthesized with the image 20 indicating that the color Doppler image is useful for diagnosis. Due to this, in the real-time display mode, the user cannot easily grasp all color Doppler images useful for diagnosis in some cases.

On the other hand, in the cine-reproduction mode, when the freeze button is pressed after the processing circuitry 140 determines that the sum of the differences in luminance is smaller than the predetermined threshold the predetermined number of times or more in a row, information indicating that the image is useful for diagnosis is generated for all the color Doppler images corresponding to the time phase in which the temporal change in luminance distribution is relatively small at the time when the freeze button is pressed. Thus, in the cine-reproduction mode, the user can easily grasp all the color Doppler images useful for diagnosis.

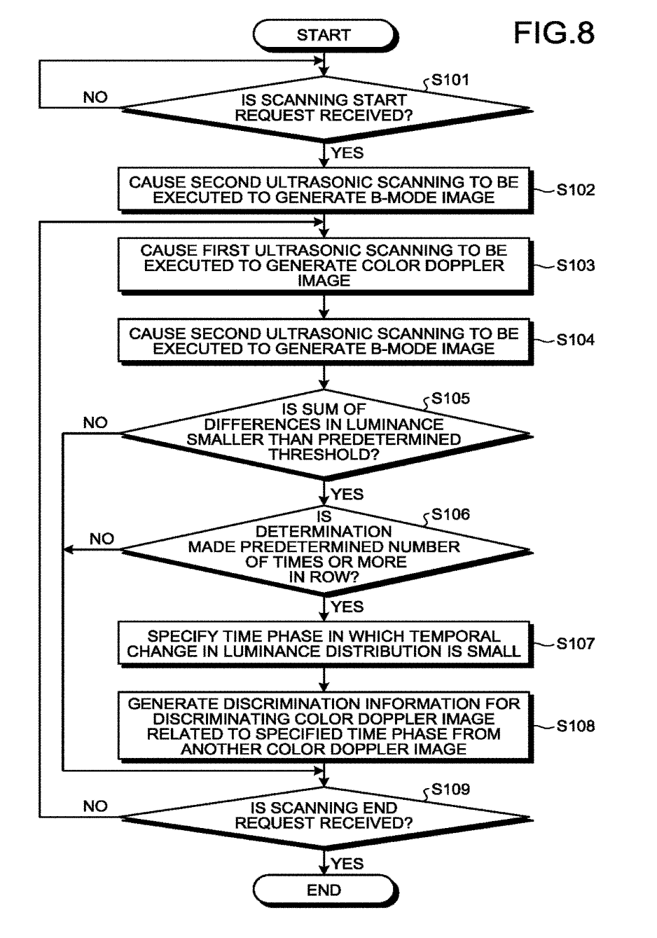

Next, the following describes an example of ultrasonic scanning control processing performed by the ultrasonic diagnostic device according to the first embodiment with reference to FIG. 8. FIG. 8 is a flowchart for explaining an example of the ultrasonic scanning control processing performed by the ultrasonic diagnostic device according to the first embodiment.

As illustrated in FIG. 8, the processing circuitry 140 determines whether a start request for ultrasonic scanning (scanning start request) is received (Step S101). If the scanning start request is not received (No at Step S101), the processing circuitry 140 performs determination at Step S101 again.

On the other hand, if the scanning start request is received (Yes at Step S101), the processing circuitry 140 causes the second ultrasonic scanning to be executed to generate the B-mode image (Step S102). The processing circuitry 140 then causes the first ultrasonic scanning to be executed to generate the color Doppler image (Step 103). The processing circuitry 140 then causes the second ultrasonic scanning to generate the B-mode image (Step S104).

The processing circuitry 140 calculates the sum of the differences in luminance between the B-mode image generated at Step S102 and the B-mode image generated at Step S104, and determines whether the calculated sum of the differences in luminance is smaller than the predetermined threshold (Step S105). If the sum of the differences in luminance is equal to or larger than the predetermined threshold (No at Step S105), the process performed by the processing circuitry 140 proceeds to Step S109 described later.

On the other hand, if the sum of the differences in luminance is smaller than the predetermined threshold (Yes at Step S105), the processing circuitry 140 determines whether the sum of the differences in luminance is determined to be smaller than the predetermined threshold the predetermined number of tunes or more in a row (Step S106). If the number of times when the sum of the differences in luminance is successively determined to be smaller than the predetermined threshold is smaller than the predetermined number (No at Step S106), the process performed by the processing circuitry 140 proceeds to Step S109 described below.

On the other hand, if the sum of the differences in luminance is determined to be smaller than the predetermined threshold the predetermined number of times or more in a row (Yes at Step S106), the processing circuitry 140 specifies, as the time phase in which the temporal change in luminance distribution is relatively small, a time phase corresponding to the B-mode image in which the sum of the differences in luminance is successively determined to be smaller than the predetermined threshold (Step S107). At Step S107, the processing circuitry 140 also specifies, as the time phase in which the temporal change in luminance distribution is relatively small, a time phase corresponding to the color Doppler image between the B-mode images in which the sum of the differences in luminance is successively determined to be smaller than the predetermined threshold.

The processing circuitry 140 generates discrimination information for discriminating the color Doppler image related to the specified time phase from another color Doppler image among a plurality of color Doppler images (Step S108). The processing circuitry 140 determines whether an end request for ultrasonic scanning (scanning end request) is received (Step S109). If the scanning end request is not received (No at Step S109), the process performed by the processing circuitry 140 returns to Step S103. If the process returns to Step S103 from Step S109, the processing circuitry 140 causes the first ultrasonic scanning to be executed to generate the color Doppler image at Step S103, causes the second ultrasonic scanning to be executed to generate the B-mode image at Step S104, and calculates the sum of the differences in luminance between the B-mode image currently generated at Step S104 and the B-mode image previously generated at Step S104 to determine whether the calculated sum of the differences in luminance is smaller than the predetermined threshold at Step S105.

On the other hand, if the scanning end request is received (Yes at Step S109), the processing circuitry 140 ends the ultrasonic scanning control processing.

The ultrasonic diagnostic device 1 according to the first embodiment has been described above. As described above, the ultrasonic diagnostic device 1 according to the first embodiment can enhance convenience for the user to perform diagnosis.

The first embodiment describes a case in which the processing circuitry 140 specifies the time phase in which movement of tissues such as a cardiac wall is gentle by specifying the time phase in which the temporal change in luminance distribution is relatively small. However, based on a similar principle, the processing circuitry 140 can also specify a time phase in which a hand shake of a tester operating the ultrasonic probe 101 is small in scanning, and a time phase in which movement of the subject P due to breathing and the like is small, by specifying the time phase in which the temporal change in luminance distribution is relatively small. The processing circuitry 140 may generate discrimination information for discriminating, from another color Doppler image, the color Doppler image corresponding to the time phase in which a hand shake of a tester in scanning is small among the generated color Doppler images. Accordingly, the user can easily grasp that which color Doppler image is useful for diagnosis like the color Doppler image corresponding to the time phase in which a hand shake of a tester in scanning is small among the generated color Doppler images. The processing circuitry 140 may generate discrimination information for discriminating, from another color Doppler image, the color Doppler image corresponding to the time phase in which movement of the subject P due to breathing and the like is small among the generated color Doppler images. Accordingly, the user can easily grasp that which color Doppler image is useful for diagnosis like the color Doppler image corresponding to the time phase in which movement of the subject P due to breathing and the like is small among the generated color Doppler images.

The first embodiment describes a case in which the transmission/reception circuitry 110 executes the second ultrasonic scanning every time when executing the first ultrasonic scanning once via the ultrasonic probe 101. However, the transmission/reception circuitry 110 may execute the second ultrasonic scanning every time when executing the first ultrasonic scanning a predetermined number of times, not once. That is, the transmission/reception circuitry 110 may execute the second ultrasonic scanning every time when executing the first ultrasonic scanning at least once.

### Modification of First Embodiment

The first embodiment describes an example in which the processing circuitry 140 specifies the time phase in which the temporal change in luminance distribution is relatively small. Alternatively, the processing circuitry 140 may specify a time phase in which the temporal change in luminance distribution is relatively large.

For example, every time the B-mode image is generated, the processing circuitry 140 determines whether the sum of the differences in luminance between the most lately generated B-mode image and the B-mode image generated immediately prior to the former B-mode image is equal to or larger than a predetermined threshold. The processing circuitry 140 determines whether the sum of the differences in luminance is determined to be equal to or larger than the predetermined threshold the predetermined number of times or more in a row. When the sum of the differences in luminance is determined to be equal to or larger than the predetermined threshold the predetermined number of times or more in a row, the processing circuitry 140 specifies, as the time phase in which the temporal change in luminance distribution is relatively large, a time phase corresponding to the B-mode image in which the sum of the differences in luminance is successively determined to be equal to or larger than the predetermined threshold. The processing circuitry 140 then generates discrimination information for discriminating the color Doppler image related to the specified time phase from another color Doppler image among a plurality of color Doppler images. For example, as the discrimination information, the processing circuitry 140 generates an image indicating a blue frame, and synthesizes the image indicating the blue frame with the color Doppler image related to the specified time phase.

Accordingly, the user can easily grasp the color Doppler image not synthesized with the blue frame as the color Doppler image useful for diagnosis. Thus, the ultrasonic diagnostic device according to the modification of the first embodiment can enhance convenience for the user to perform diagnosis.

### Second Embodiment

The first embodiment describes a case in which the processing circuitry 140 generates the image indicating the red frame as the discrimination information. However, the discrimination information is not limited thereto. As the discrimination information, the processing circuitry 140 may generate a mark serving as an indicator for causing the user to easily grasp the color Doppler image is useful for diagnosis. Such an embodiment will be described as a second embodiment.

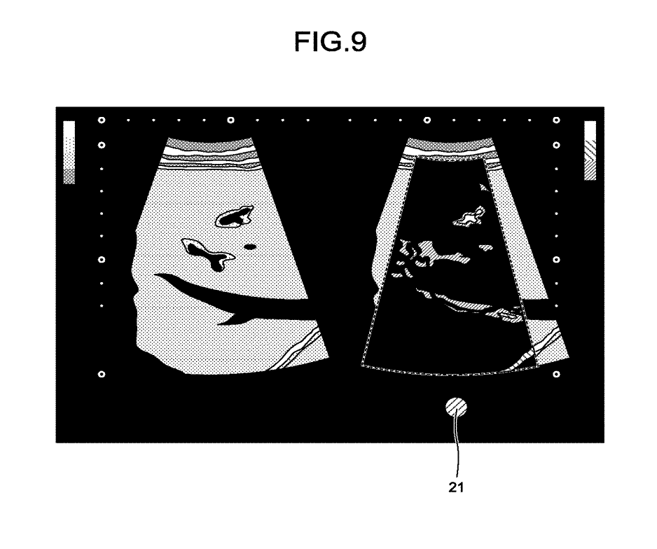

FIG. 9 is a diagram for explaining an example of discrimination information according to the second embodiment. The processing circuitry 140 according to the second embodiment generates the mark described above as the discrimination information. As illustrated in the example of FIG. 9, the processing circuitry 140 synthesizes a mark 21 with the color Doppler image related to the time phase in which the temporal change in luminance distribution is relatively small among a plurality of color Doppler images, and causes the display 103 to successively display a plurality of color Doppler images including the color Doppler image to which the mark 21 is synthesized in accordance with an operation of the trackball, the dial or the like by the user. Accordingly, the user can easily grasp the color Doppler image to which the mark 21 is synthesized as the color Doppler image useful for diagnosis. Thus, the ultrasonic diagnostic device according to the second embodiment can enhance convenience for the user to perform diagnosis.

### Third Embodiment

The processing circuitry 140 may generate, as the discrimination information, a graphic for notifying the user that the color Doppler image is useful for diagnosis. Such an embodiment will be described as a third embodiment.

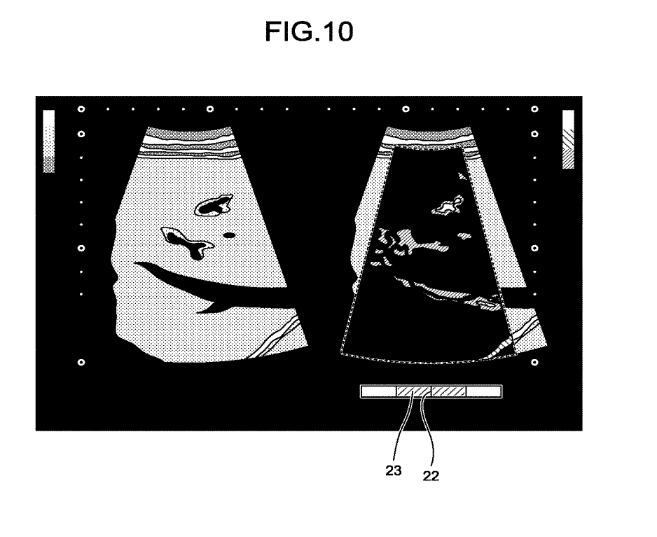

FIG. 10 is a diagram for explaining an example of discrimination information according to the third embodiment. In the example of FIG. 10, described is a case in which the color Doppler image in accordance with an operation of the trackball, the dial or the like by the user is displayed from among a plurality of color Doppler images. In the example of FIG. 10, illustrated is a bar 22 indicating a generation order of the color Doppler image being displayed on the display 103. That is, a position of the bar 22 is moved to a position corresponding to the generation order of a newly displayed color Doppler every time the color Doppler image displayed on the display 103 is switched.

The processing circuitry 140 according to the third embodiment specifies the position of the bar 22 (smallest order position) in a case in which the color Doppler image the generation order of which is the smallest is displayed on the display 103 from among a plurality of color Doppler images related to the time phase in which the temporal change in luminance distribution is relatively small, that is, a plurality of color Doppler images useful for diagnosis. The processing circuitry 140 specifies the position of the bar 22 (largest order position) in a case in which the color Doppler image the generation order of which is the largest is displayed on the display 103 from among a plurality of color Doppler images useful for diagnosis. As illustrated in the example of FIG. 10, the processing circuitry 140 generates a graphic 23 in a range from the smallest order position to the largest order position. The graphic 23 represents the generation order of the color Doppler image useful for diagnosis.

Thus, when the bar 22 is positioned on the graphic 23, the color Doppler image being displayed is the color Doppler image useful for diagnosis. Due to this, the ultrasonic diagnostic device according to the third embodiment can cause the user to easily grasp that the color Doppler image being displayed is the color Doppler image useful for diagnosis by causing the user to easily grasp that the bar 22 is positioned on the graphic 23. Accordingly, the ultrasonic diagnostic device according to the third embodiment can enhance convenience for the user to perform diagnosis.

### Fourth Embodiment

The first embodiment to the third embodiment describe a case in which the processing circuitry 140 specifies, as the time phase in which the temporal change in luminance distribution of the B-mode image is relatively small, the time phase corresponding to the B-mode image in which the sum of the differences in luminance is determined to be smaller than the predetermined threshold the predetermined number of times or more in a row, and generates the discrimination information for discriminating the color Doppler image related to the specified time phase from another color Doppler image. However, the processing circuitry 140 does not necessarily specify the time phase. Such an embodiment will be described as a fourth embodiment.

For example, in the fourth embodiment, the processing circuitry 140 treats, as the time phase in which the temporal change in luminance distribution of the B-mode image is relatively small, a time phase corresponding to the B-mode image in which the sum of the differences in luminance is determined to be smaller than the predetermined threshold the predetermined number of times or more in a row. The processing circuitry 140 generates discrimination information for discriminating, from another color Doppler image, the color Doppler image related to the time phase in which the temporal change in luminance distribution of the B-mode image is relatively small. In this way, in the fourth embodiment, the processing circuitry 140 generates the discrimination information without specifying the time phase.

### Fifth Embodiment

Next, the following describes the ultrasonic diagnostic device 1 according to a fifth embodiment. The first embodiment describes a case in which, in the cine-reproduction mode, the processing circuitry 140 acquires a plurality of B-mode images and color Doppler images stored in the image memory 150, and reproduces the B-mode images and the color Doppler images as moving images on the display 103 in accordance with the rotational direction, rotation amount, and the like of the trackball, the dial or the like operated by the user.

However, the processing circuitry 140 may reproduce only the color Doppler image related to the time phase in which the temporal change in luminance distribution of the B-mode image is relatively small on the display 103 from among a plurality of color Doppler images stored in the image memory 150 in the cine-reproduction mode. That is, in the cine-reproduction mode, the processing circuitry 140 may successively cause the color Doppler image to be displayed, the color Doppler image being related to the time phase in which the temporal change in luminance distribution of the B-mode image is relatively small, from among a plurality of color Doppler images stored in the image memory 150, in accordance with the rotation of the trackball, the dial, or the like or automatically. In this way, the processing circuitry 140 may cause the color Doppler image to be displayed, the color Doppler image being related to the time phase in which the temporal change in luminance distribution of the B-mode image is relatively small, while skipping the color Doppler image related to the time phase in which the temporal change in luminance distribution of the B-mode image is relatively large. Similarly, in the cine-reproduction mode, the processing circuitry 140 may reproduce the B-mode image in which the temporal change in luminance distribution is relatively small on the display 103 from among a plurality of B-mode images stored in the image memory 150. That is, the processing circuitry 140 may cause the B-mode image in which the temporal change in luminance distribution is relatively small to be displayed while skipping the B-mode image in which the temporal change in luminance distribution is relatively large.

Accordingly, the user can grasp only the color Doppler image useful for diagnosis from among a plurality of color Doppler images stored in the image memory 150. Thus, convenience for the user to perform diagnosis can be enhanced.

### Sixth Embodiment

Next, the following describes the ultrasonic diagnostic device 1 according to a sixth embodiment. In the sixth embodiment, the processing circuitry 140 performs a maximum value holding arithmetic operation related to an index value indicating blood flow information on a plurality of color Doppler images to generate an image indicating a region in which a blood flow of the subject P is present, and causes the display 103 to display the image. The index value indicating the blood flow information is, for example, a value of power.

The maximum value holding arithmetic operation is, for example, an arithmetic operation of selecting the maximum value from among luminance values (values of power) assigned to a spatially corresponding pixel in each color Doppler image, and generating a new image using the selected maximum value as the value of power for the corresponding pixel.

In the sixth embodiment, the processing circuitry 140 performs the maximum value holding arithmetic operation on not all the generated color Doppler images, and performs the maximum value holding arithmetic operation on the color Doppler image related to the time phase in which the temporal change in luminance distribution of the B-mode image is relatively small.

For example, the processing circuitry 140 selects a plurality of color Doppler images, performs the maximum value holding arithmetic operation on the selected color Doppler images, generates one image indicating a region in which the blood flow of the subject P is present, and causes the display 103 to display the image. In this case, one static image is displayed on the display 103.

After causing the display 103 to display one image indicating the region in which the blood flow of the subject P is present, the processing circuitry 140 selects the maximum value of the value of power in the newly generated color Doppler image every time the color Doppler image is generated, and may update the image indicating the region in which the blood flow of the subject P is present using the selected maximum value of the value of power. In this case, the image indicating the region in which the blood flow of the subject P is present displayed on the display 103 is successively updated to be reproduced.

In this way, the processing circuitry 140 performs maximum value holding arithmetic operation on the color Doppler image generated based on the reflected wave data collected in the time phase in which movement of tissues is gentle. That is, the processing circuitry 140 selects the maximum value of the value of power in the color Doppler image in which movement of tissues is gentle without selecting the maximum value of the value of power in the color Doppler image in which movement of tissues is large, and generates the image indicating the region in which the blood flow of the subject P is present.

Thus, according to the sixth embodiment, generated is an image in which visibility is improved not only for a relatively large blood flow but also for tissue perfusion at a capillary level.

The ultrasonic scanning control processing described above in the embodiments can be implemented by executing an ultrasonic scanning control processing program prepared in advance using a computer such as a personal computer and a workstation. The ultrasonic scanning control processing program can be distributed via a network such as the Internet. The ultrasonic scanning control processing program can be recorded in a computer-readable recording medium such as a hard disk, a flexible disk (FD), a CD-ROM, an MO, and a DVD, and executed by being read out from the recording medium by a computer.

According to at least one of the embodiments described above, convenience for the user to perform diagnosis can be enhanced.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic device comprising:
transmission/reception circuitry configured to
intermittently execute first ultrasonic scanning on a first scanning region of a subject in accordance with a first scanning condition for generating a color Doppler image, and
intermittently execute second ultrasonic scanning on a second scanning region of the subject in accordance with a second scanning condition for generating a form image; and
processing circuitry configured to
generate a plurality of color Doppler images based on first echo data collected through the first ultrasonic scanning,
generate a plurality of form images based on second echo data collected through the second ultrasonic scanning, and
generate discrimination information for discriminating, from another color Doppler image, a color Doppler image related to a time phase in which a temporal change in luminance distribution of each of the form images is relatively large or a time phase in which the temporal change is relatively small.

2. The ultrasonic diagnostic device according to claim 1, wherein the processing circuitry synthesizes part of the color Doppler images with an indicator based on the discrimination information, and causes the color Doppler images to be successively displayed, the color Doppler images including the color Doppler image to which the indicator is synthesized.

3. The ultrasonic diagnostic device according to claim 1, wherein the processing circuitry causes part of the color Doppler images to be highlighted based on the discrimination information.

4. The ultrasonic diagnostic device according to claim 1, wherein the processing circuitry specifies the time phase in which the temporal change in luminance distribution of each of the form images is relatively large or the time phase in which the temporal change is relatively small, generates a graphic representing a generation order of a color Doppler image related to a specified time phase based on the discrimination information, and causes a display to display the generated graphic.

5. The ultrasonic diagnostic device according to claim 1, wherein the transmission/reception circuitry executes the second ultrasonic scanning every time the first ultrasonic scanning is executed at least once.

6. The ultrasonic diagnostic device according to claim 1, wherein the second scanning condition is different from the first scanning condition in at least one of a frequency band of ultrasonic waves to be transmitted and a frequency band of ultrasonic waves to be received.

7. The ultrasonic diagnostic device according to claim 1, wherein the second scanning region is smaller than the first scanning region.

8. The ultrasonic diagnostic device according to claim 1, wherein the second scanning region is larger than the first scanning region.

9. The ultrasonic diagnostic device according to claim 1, wherein the first ultrasonic scanning is ultrasonic scanning in which ultrasonic waves are transmitted and received once for each scanning line in the first scanning region including a plurality of scanning lines.

10. The ultrasonic diagnostic device according to claim 1, further comprising:
storage circuitry configured to store the plurality of color Doppler images generated by the processing circuitry, wherein
the processing circuitry successively displays a color Doppler image related to the time phase in which the temporal change in luminance distribution of each of the form images is relatively small from among the plurality of color Doppler images stored in the storage circuitry.

11. The ultrasonic diagnostic device according to claim 1, wherein the processing circuitry performs a maximum value holding arithmetic operation related to an index value indicating blood flow information on a color Doppler image related to the time phase in which the temporal change in luminance distribution of each of the form images is relatively small.

12. The ultrasonic diagnostic device according to claim 1, wherein
the processing circuitry
specifies the time phase in which the temporal change in luminance distribution of each of the form images is relatively large or a time phase in which the temporal change is relatively small based on the form images, and
generates the discrimination information for discriminating a color Doppler image related to the specified time phase from another color Doppler image.

13. A method of generating discrimination information, the method comprising, by processing circuitry connected to a transmission/reception circuitry that is configured to intermittently execute first ultrasonic scanning on a first scanning region of a subject in accordance with a first scanning condition for generating a color Doppler image, and configured to intermittently execute second ultrasonic scanning on a second scanning region of the subject in accordance with a second scanning condition for generating a form image:
generating a plurality of color Doppler images based on first echo data collected through the first ultrasonic scanning,
generating a plurality of form images based on second echo data collected through the second ultrasonic scanning, and
generating discrimination information for discriminating, from another color Doppler image, a color Doppler image related to a time phase in which a temporal change in luminance distribution of each of the form images is relatively large or a time phase in which the temporal change is relatively small.
